(19) United States
(12) Patent Application Publication
(10) Pub. No.: US 2013/0108677 A1
(43) Pub. Date: May 2, 2013

(54) ABRASIVE CONFECTIONARY PRODUCTS

(71) Applicant: BREEZY INDUSTRIES LTD., Kiryat Arba (IL)

(72) Inventor: Shimon Harpaz, Netanya (IL)

(73) Assignee: BREEZY INDUSTRIES LTD., Kiryat Arba (IL)

(21) Appl. No.: 13/717,598

(22) Filed: Dec. 17, 2012

Related U.S. Application Data
(63) Continuation of application No. PCT/IL2011/000478, filed on Jun. 16, 2011.
Application No. PCT/IL2011/000478 is a continuation-in-part of application No. 12/663,581, filed on Dec. 28, 2009.
(60) Provisional application No. 61/355,570, filed on Jun. 17, 2010.

(30) Foreign Application Priority Data
Jun. 10, 2007 (IL) 183818

(51) Int. Cl. A61K 8/11; A61Q 11/00

(52) U.S. Cl. CPC A61K 8/11; A61Q 11/00
USPC 424/401; 424/49

(57) ABSTRACT

There is provided a consumable abrasive confectionery product for the reduction of oral malodor and for treatment of other oral cavity conditions, comprising substantially uniformly dispersed abrasive particles configured to at least partially erode deposits in the oral cavity, wherein the abrasive quality of the confectionery product is substantially constant as the confectionery product is consumed and wherein the use of the product does not induce a gag reflex.

### CROSS-REFERENCE TO RELATED APPLICATIONS

This application is a Continuation of International Application No. PCT/IL2011/000478, filed Jun. 16, 2011, which claims the benefit of U.S. Provisional Application No. 61/355,570, filed Jun. 17, 2010, and is also a Continuation-In-Part of U.S. patent application Ser. No. 12/663,581, filed Dec. 8, 2009, which is the U.S. National Stage of International Application No. PCT/IL2008/000783, filed Jun. 10, 2008, which claims priority to Israeli Application No. 183818, filed Jun. 10, 2007, the contents of each of which are herein expressly incorporated by reference for all purposes.

### FIELD OF THE INVENTION

The invention relates to abrasive confectionery products. Some embodiments of the invention relate to confectionery products comprising abrasive particles for treating oral malodor and other oral cavity conditions.

### BACKGROUND OF THE INVENTION

Oral malodor is a generic term used to describe foul smell emanating from the mouth (oral cavity). One of the sources of mouth and oral malodor is deposits of bacteria, including anaerobic bacteria, on the tongue, such as on the posterior dorsum of the tongue. The bacteria can thrive on remnants of food deposits, dead epithelial cells, postnasal drip or on other bacteria. The anaerobic respiration of the bacteria can yield putrescent smell of compounds such as indole, skatole, polyamines, or the smell of volatile sulfur compounds (VSC) such as hydrogen sulfide, methyl mercaptan, allyl methyl sulfide or dimethyl sulfide (for example, Rosenberg M., Clinical assessment of bad breath: current concepts. J Am Dent Assoc. 1996 April; 127(4):475-82). Additional causes of oral malodor include physiological processes and behaviors such as, for example, hunger, smoking, prescription drugs, and the like.

Various methods are known for the assessment of oral malodor. For example, one assessment of oral malodor uses the human sense of smell (organoleptic method) and is measured on a 0-5 scale in order to consistently quantify the odor. Oral malodor may also be analyzed using gas chromatography coupled with flame photometric detection, allowing for separation and quantitative measurements of the individual gasses. However, this method is costly, time-consuming and the equipment used is cumbersome. Portable sulfide meters are also widely used, but despite being portable and relatively easy to use, the presence of certain chemicals found in perfume, deodorants and mouthwashes, interfere with their operation.

It has been shown that tongue brushing and tooth brushing yielded a 70% and 30% reduction, respectively, in measurements of oral malodor using organoleptic analysis (Tonzetich and Ng, Reduction of malodor by oral cleansing procedures. Oral Surg Oral Med Oral Pathol 42: 172-181 (1976)). Hence, the removal or controlled reduction of bacteria by mechanical or chemical means can significantly reduce oral malodor.

The dorsoposterior third of the tongue (dorsal region) is not constantly scraped clean through mechanical interaction with the hard plate and teeth, unlike the anterior two thirds, and moreover this area is also missed by direct brushing, mouthwashes and other means of cleaning the oral cavity as it may trigger a gag reflex. In addition, the saliva functions as an antibacterial, antiviral and antifungal buffering and cleaning agent, so any treatment that increases saliva flow and tongue movement, such as sucking or chewing on a confectionery sweet, may further aid in reducing oral malodor.

Confectionery products for addressing oral malodor are disclosed in prior publications. Some of these products provide abrasive surfaces which are intended for scraping the tongue, thereby dislodging microorganisms and their metabolites which contribute to malodor. Some publications are cited below as examples.

U.S. Pat. No. 6,004,334 relates to a tongue cleaning apparatus comprising a lozenge-shaped edible dissolvable candy having two sides of different compositions adhered by an edible adhesive. One side is a hard candy material having a pattern formed in it which is suitable for abrading the tongue and the other side is a soft pliable candy material having a depression formed in its center to allow it to adhere by suction to the roof of the mouth.

U.S. Pat. No. 7,063,858 relates to a boiled sugar sweet with a rough texture intended for treating halitosis, comprising a crystalline ingredient, such as a powdered saccharide or an organic acid, wherein the crystalline ingredient exhibits a particle size of greater than 200 microns and a melting point of greater than 110° C.

US application 2003/0163149 relates to a breath freshener lollipop in which the edible applicator head has a textured surface and includes an antibacterial medicament for eliminating volatile sulfur compound precursors on the tongue. Medicaments disclosed are divalent elements, such as zinc or copper.

US application 2006/0222683 relates to a soft pliable dissolvable candy which provides tongue scraping action. According to the disclosure, a plurality of hard candy segments, preferably having raised ridges, is formed in one surface of the soft candy, and a plurality of depressions is formed in another side of the soft candy.

International application WO2005/102066 and US application 2006/0193909 relate to a pressed tablet having at least one abrasive surface suitable for cleaning the surface of the tongue. One related product has different layers such that the surface of one layer is smooth and the surface of the other layer is abrasive, wherein the abrasive surface is formed with abrasive structure and/or comprises abrasive particles embedded in a layer. Another related product is a pressed tablet formed of one composition and having abrasive inclusions wherein the latter comprise solid matrices, for example, carbohydrates or polyols, and may further include encapsulated or entrapped flavors and colors.

US application 2007/0054014 relates to a confectionery product for cleaning the surface of the tongue, the product comprising a first side and a second opposite side wherein the second side comprises an abrasive surface, and wherein the second side has a width and a length, the smallest of which is at least 1.6 times the product thickness. A related product comprises abrasive particles incorporated into the composition, or located on the abrasive surface, or embedded in the abrasive surface. Generally, the particles are hard particles of at least 100 microns which are less soluble than the surrounding matrix, and may be made from solid matrices and extruded carbohydrates or polyols, and may further include encapsulated or entrapped flavors and colors.

U.S. Patent Application Publication No. 2007/0134168 discloses a chewable oral composition for freshening the breath, such as a chewable candy, the composition comprising: (a) a water soluble bulk portion; (b) a gum base portion; and (c) a coating layer including fast release antimicrobial agent comprising a synergistic ratio of Magnolia Bark Extract and surface active agent. The disclosed antimicrobial agent may be encapsulated in a coated matrix or a biodegradable polymeric matrix.

U.S. Pat. No. 6,602,518 discloses a product including a medicament comprising: a consumable center, such as a hard center; and a compressible composition comprising an encapsulated medicament that is compressed around the consumable center. U.S. Pat. No. 5,370,864 discloses a microcapsule suitable for reducing oral bacteria and providing breath protection comprising a shell material and a core composition.

International Patent Application Publication No. WO 2008/152626, to the inventor of the present invention, describes an abrasive confectionery product and a process for producing the same.

U.S. Pat. No. 4,933,190 discloses a sweetener delivery system for incorporation into a chewing gum composition, the sweetener delivery system comprising a first coating formed from the combination of an emulsifier with low molecular weight polyvinyl acetate and a second outer coating prepared from a hydrophilic polymer. Such prior art products may mask breath malodor but do not address removal of tongue coat substances which are the source of the malodor. There is thus a need in the art for an abrasive confectionery product that by simple and everyday use can provide enhanced oral cavity cleaning abilities, which in turn results in enhanced diminishing and/or elimination of oral malodor, and in addition may be used to attend other oral cavity conditions, such as, for example, candidiasis, various infections, burning mouth syndrome, xerostomia, abscess, and the like.

### SUMMARY OF THE INVENTION

According to some embodiments there is provided a confectionery abrasive product that is able to provide deep cleaning of surfaces in the oral cavity (such as, for example, the inner grooves of the tongue), does not induce a gagging reflex and can substantially maintain its abrasive quality (abrasive ability) throughout its consumption cycle. Maintaining the abrasive quality over time is advantageous as it provides a longer lasting abrading activity at all time points of its use and hence provides an improved result in diminishing or eliminating oral malodor and may also be used in treating additional oral cavity conditions, such as, for example, candidiasis, bacterial and viral infections, burning mouth syndrome, xerostomia, abscess, gum inflammation, tooth decay, mouth ulcers, aphtha, and the like. In addition, the abrasive particles, which are substantially uniformly dispersed within the confectionery product and are adapted to provide deeper abrading and cleaning properties by penetrating into the inner grooves and folds of the tongue augment the beneficial effect on oral malodor, by reaching hard to reach inner deposits. Moreover, the particles may further include one or more ingredients that may include health and/or oral enhancing agents that may further provide beneficial effect on a subject's health in general and on oral malodor and other oral cavity related conditions in particular. Furthermore, the absence of gagging reflex when using the confectionery product enables cleaning in hard to reach deposits located in regions that would otherwise be inaccessible because of the gagging reflex.

In some embodiments the particles may have any geometrical shape and form. The particles have coarse or prickly or rough surfaces optionally with sharpened or pointed edges. In some embodiments, the particles may have smooth surfaces and abrade deposits on the tongue due to the hardness of the composition of the particles relative to the composition of the deposits. Advantageously, due to the shape and size of the particles, the particles are able to provide deep tongue cleaning, as they may penetrate below the film of the tongue and able to penetrate the inner grooves and folds of the tongue. Such deep cleaning provides an enhanced and superior abrading of deposits of the tongue and results in an improved cleaning of the tongue and enhanced reduction of oral malodor as compared to superficial abrading of the tongue surface.

According to some embodiments, it is advantageous to have the confectionery abrasive product in the form of a cylinder or cubical/rectangular, so as to provide deeper abrading and cleaning of the oral cavity by providing improved access to various regions of the oral cavity.

According to some embodiments, there is thus provided a consumable abrasive confectionery product adapted to reduce oral malodor, comprising substantially uniformly dispersed abrasive particles configured to at least partially erode, scrub, loosen and/or abrade deposits in the oral cavity, wherein the abrasive quality of the confectionery product is substantially constant as the confectionery product is consumed.

According to additional embodiments, there is provided a consumable abrasive confectionery product adapted to treat oral cavity related condition, comprising substantially uniformly dispersed abrasive particles configured to at least partially erode, scrub, loosen and/or abrade deposits in the oral cavity, wherein the abrasive quality of the confectionery product is substantially constant as the confectionery product is consumed. In some embodiments, the abrasive quality is in the range of about 10-25 gr/cm². In some embodiments, the abrasive quality is in the range of about 13-18 gr/cm². In further embodiments, the density of the abrasive particles is in the range of about 0.1-2 gr/ml. In some embodiments the density of the abrasive particles is in the range of about 0.1-1 gr/ml.

In some embodiments, the confectionery product comprises an elongated stick partly disposed inside the confectionery product about an axis. Preferably, the stick has an appropriate cross-section perpendicular to the length thereof, at least along a part of the portion outside the confectionery product. The stick may include a safety stick, a toothpick, and the like.

In embodiments of the invention, the confectionery product comprises substantially uniformly dispersed abrasive particles, such as crystalline compounds. In some embodiments, the particles are formed as granules or pellets or corpuscles optionally with coarse surfaces and/or sharpened and/or extending edges. Additionally or alternatively, the particles are capsules comprising an inner core and an external coating or shell, wherein the shell and/or core are suitably hard and/or coarse to abrade and/or erode the deposits on the tongue. Preferably, the particles dissolve and/or disintegrate in the oral cavity and/or the gastrointestinal tract.

In some embodiments of the invention, the particles sizes (e.g. maximal or average diameter) are small such as up to about 1000 μm. For example, the particles sizes are dispersed in a range between about 50 μm to about 1000 μm or a sub-range thereof. In some embodiments, the particles sizes are dispersed in a range between about 50 μm to about 850 μm. In some embodiments, the particles are larger than about 1000 microns, and the particles sizes are dispersed in a range between about 1000 micron to about 2500 micron. In some embodiments, the size distribution and/or concentration/density and/or composition of the dispersed particles is, at least approximately, uniform throughout the confectionery product. According to some embodiments, the density of the particles is in the range of about the range of about 0.1-2 gr/ml. In some embodiments, the concentration of the particles is about 0.1-5% of total weight of the product.

In some embodiments of the invention, the size distribution and/or concentration and/or composition of the dispersed particles are non-uniform and varies through and/or between different parts of the confectionery product such as to provide different degrees of abrading. For example, spatial variation is in concentric rings, or in layers parallel to the axis of the shape, or along the length of the confectionery product such as to provide enhanced abrasion in the base of the tongue (posterior dorsal part) where the malodor producing deposits are likely to be found, relative to other parts of the tongue.

In some embodiments of the invention, the confectionery product is hard and/or rigid and/or firm such as or similar to a gel of partially crystallized compositions or a solid solution (or mixture) of crystallized composition with softer or fluid material such as water. In other embodiments of the invention, the confectionery product is flexible and/or semi-hard, for example, such as toffee or rubber.

With a flexible confectionery product, various parts in the oral cavity, in addition to the tongue, may be abraded, such as, for example, plaque off the gums or teeth, thereby providing extended functionality as a mouth hygiene tool. Likewise, the tip of the stick outside the confectionery product may be sharpened, providing an integral toothpick. Optionally, the other end of the stick is also sharpened, providing a toothpick after the confectionery product is consumed or spread out in order to prevent dislodging.

According to some embodiments, there is provided a consumable abrasive confectionery product adapted to reduce oral malodor comprising substantially uniformly dispersed abrasive particles configured to at least partially erode, scrub, loosen and/or abrade deposits in the oral cavity, wherein the abrasive quality of the confectionery product is substantially constant as the confectionery product is consumed.

According to some embodiments, the confectionery product may further include a stick partially disposed in the confectionery product. The stick may be a safety stick, a tooth pick, or both.

In some embodiments, the oral cavity may include the tongue, cheek, palate, teeth, or any combination thereof.

In additional embodiments, the particles may be structured to at least partially erode, scrub, loosen and/or abrade deposits on the inner grooves of the tongue. The particles may further be structured to at least partially erode, scrub, loosen and/or abrade deposits on dorsal region of the tongue.

In some embodiments, the particles may include granules, capsules, or both. The capsules may include an external shell and an internal core. The particles may include ingredients, which may include at least one of an inert substance, a medication, beneficial composition, freshener, fragrance or an active compound for reacting with materials in deposits in the oral cavity. In some embodiments, the active compound may be a compound of a divalent metal, such as Zinc. In some embodiments, the inert substance is tricalcium phosphate (TCP). In further embodiments, the capsules may include a combination of Zinc and TCP. In some embodiments, the particles may adapted for a slow release at least in one of the oral cavity or gastrointestinal tract of the ingredient comprised in the particles.

According to further embodiments, the size of a particle may be up to about 1 millimeter. In some embodiments, the particles size is distributed between about 50 μm to about 1000 μm. The particles may be similar in size and/or shape. In some embodiments, the particles may be different in size and shape.

According to yet further embodiments, the confectionery product may not induce a gag reflex when inserted into the oral cavity.

### BRIEF DESCRIPTION OF THE DRAWINGS

Some non-limiting exemplary embodiments of the invention are illustrated in the following drawings.

Identical or duplicate or equivalent or similar structures, elements, or parts that appear in one or more drawings are generally labeled with the same reference numeral, optionally with an additional letter or letters to distinguish between similar objects or variants of objects, and may not be repeatedly labeled and/or described.

Dimensions of components and features shown in the figures are chosen for convenience or clarity of presentation and are not necessarily shown to scale or true perspective. For convenience or clarity, some elements or structures are not shown or shown only partially and/or with different perspective or from different point of views.

FIG. 1A-1F schematically illustrates exemplary perspective view of a confectionery product, according to some embodiments;

FIGS. 1A-1K schematically illustrates exemplary cross section views of confectionery products, according to some embodiments

FIG. 2A schematically illustrates a perspective view of an abrasive confectionery product comprising a bulk with dispersed abrasive particles, according to exemplary embodiments of the invention;

FIG. 2B schematically illustrates an enlarged view of a sample of the confectionery product of FIG. 2A with abrasive particles as granules, according to exemplary embodiments of the invention;

FIG. 2C schematically illustrates an enlarged view of a sample of the confectionery product of FIG. 2A with abrasive particles as capsules, according to exemplary embodiments of the invention;

FIG. 2D schematically illustrates an enlarged view of a sample of the confectionery product of FIG. 2A with abrasive particles as a mixture of granules and capsules, according to exemplary embodiments of the invention;

FIG. 3A schematically illustrates a perspective view of an abrasive confectionery product having dispersed abrasive particles in concentric rings, each with different concentration, according to exemplary embodiments of the invention;

FIG. 3B schematically illustrates a perspective view of an abrasive confectionery product having dispersed abrasive particles in regions parallel to the symmetry axis of the confectionery product, each with different concentration, according to exemplary embodiments of the invention;

FIG. 3C schematically illustrates a perspective view of an abrasive confectionery product having dispersed abrasive particles in regions perpendicular to the symmetry axis of the confectionery product, each with different concentration, according to exemplary embodiments of the invention;

FIG. 3D schematically illustrates a perspective view of an abrasive confectionery product having dispersed abrasive particles in continuously varying concentrations in regions perpendicular to the symmetry axis of the confectionery product, according to exemplary embodiments of the invention;

FIG. 4A schematically illustrates a perspective view of an abrasive confectionery product having a stick with sharpened end outside the confectionery product, according to exemplary embodiments of the invention; and

FIG. 4B schematically illustrates a perspective view of an abrasive confectionery product having a stick with sharpened ends both outside and inside the confectionery product, according to exemplary embodiments of the invention.

FIG. 5 shows a graph illustrating percent reduction of malodor over time of various experimental groups, as determined by organoleptic scoring.

FIG. 6 shows a graph illustrating percent reduction of malodor over time of various experimental groups as determined by Halimetric readings.

### DESCRIPTION OF EMBODIMENTS OF THE INVENTION

The following description relates to one or more non-limiting examples of embodiments of the invention. The invention is not limited by the described embodiments or drawings, and may be practiced in various manners or configurations or variations. The terminology used herein should not be understood as limiting unless otherwise specified.

In the specification and claims the following terms and derivatives and inflections thereof imply the respective non-limiting characterizations below.

Upper/Lower, Top/Bottom (side of a confectionery product)—for a confectionery product with a stick at one side, the lower (bottom) side is nearest to the stick and the upper (top) side is the opposite side.

Sugar—a representative composition of a confectionery product, representing particular sugars such as sucrose of fructose or a mixture of sugars, optionally with other compositions such as flavors. Yet, the term sugar also represents substitutes such as sugar alcohols, isomalt for example, optionally with other compositions such as sucralose.

As used herein, the term “abrasive” refers to the ability of an abrasive particle/product to scrub and/or erode and/or abrade and/or loosen and/or remove deposits (such odor causing substances) from various surfaces.

The term “abrasive quality” relates to the abrasive parameters of an abrasive product. Abrasive parameters may include such parameters as, but not limited to: abrasive force, abrasive friction, ability of erode deposits from a surface, ability to loosen deposits from a surface, ability to scrub deposits from a surface, ability to abrade deposits from a surface, ability to remove deposits from a surfaces, and the like. In some embodiments, the abrasive quality may be measured in units of gr/cm². In some embodiments, the abrasive quality may be measured in units of kN (kilo Newtons) and/or kPa (kilo Pascals).

The terms “particles” and “abrasive particles” may interchangeably be used. The terms relate to abrasive inclusions, dispersed throughout out the confectionery product, having the ability to at least partially erode and/or abrade and/or scrub and/or remove and/or loosen deposits and substances from surfaces and grooves in the oral cavity. Surfaces and grooves in the oral cavity, include, for example, but not limited to: the surface and grooves of the tongue, in particular in the dorsal region, the cheeks, the palate, the gum, teeth, and the like. The abrasive particles are not required to be of uniform shape or size. The particles may include coarse surfaces and/or sharpened and/or extending edges and may be at any geometrical shape. In some embodiments, the particles may dissolve and/or disintegrate in the oral cavity and/or the gastrointestinal tract

Granules—a type of abrasive particles having at least approximate contiguous composition throughout the volume thereof, including pellets and/or grains.

Capsules—a type of particles having a volume divided to at least an external outermost shell or coating or layer and an inner core, each having a different composition and/or state (e.g. solid vs. liquid).

Size (of particle)—taken, at least approximately, as the maximal diameter (or longest cord), or as the average diameter such as assuming the same mass and texture as a circle or a sphere (according to the shape). Optionally, other practical measures of the size are used such as a derived from the weight and/or volume of particles. The particles are not limited in size. For example, the size of the particles may be in the range of 0.1-1000 micron. For example, the size of the particles may be in the range of 50-850 micron. In some embodiments, the size of the particles may be over 1000 microns. For example, the size of the particles may be in the range of 1000-2500 micron. For example, the size of the particles may be in the range of 1000-2000 micron.

The term “deposits” relates to various deposits in the oral cavity. Deposits may include various substances such as, for example, food debris, chemical molecules, bacteria and/or metabolism products thereof, contaminations, and the like, or any combination thereof, that may reside in or on surfaces in the oral cavity.

As used herein the term “substantially uniformly dispersed throughout said base material” and related grammatical constructions mean that the distribution of the abrasive particles within the base material is substantially even or similar in all regions therein and that there is no region in which the proportion of abrasive inclusions is substantially greater or lesser compared to any other region therein.

The term “base material” or “base confectionery material” are used herein interchangeably to refer to the confectionery product material which forms the bulk of the confectionery product disclosed herein and is the medium in which the abrasive inclusions are substantially uniformly dispersed. The base material may be composed of glucose or sucrose as in standard confectionery material or may comprise “reduced-calorie” and “sugar-free” material substitutes known in the art such as sorbitol, xylitol and the like.

As used herein, the term “dissolvable” and related grammatical forms mean that a substance characteristically changes from a solid state to a liquid or semi-liquid state upon exposure to particular conditions (such as, for example, temperature range pH range, mechanical force, and the like), either in the presence or absence of a liquid.

The terms “density” and “concentration” may interchangeably be used and are directed to the number/weight of particles per a given volume. For example, the density/concentration may be measured in units of gr/ml.

As used herein, the term “oral cavity” relates to a region (space) whose boundaries are defined by the lips, cheeks, hard and soft palates, and glottis. The oral cavity includes various parts and regions within the cavity of the mouth, such as, for example, but not limited to: various parts of the tongue, teeth, gums, soft palate, hard palate, chicks, and the like.

As used herein, the term “oral cavity related condition” and “oral cavity condition” may interchangeably be used and are directed to any disease, state and/or syndrome related to the oral cavity. Exemplary oral cavity related conditions include such conditions as, but not limited to: oral malodor (halitosis), candidiasis, various infections (such as, bacterial, fungal and viral) in the oral cavity, lesions, abscess, ulcers, burning mouth syndrome, xerostomia, aphtha, gum diseases, tooth decay, and the like.

As used herein, the term “treating” with respect to oral cavity related condition is directed to reducing, ameliorating and/or eliminating the oral cavity related condition and/or the symptoms associated with the oral cavity condition.

The non-limiting section headings used herein are intended for convenience only and should not be construed as limiting the scope of the invention.

### Basic Shapes

According to some embodiments, the confectionery product may be at any shape and size that is useful and comfortable for use within the oral cavity. For example, the confectionery product may be symmetrical or non symmetrical. The confectionery product may have round edges, rough edges, soft edges or any combination thereof. The confectionery product may include a stick (such as, for example, a safety stick), used to hold and move the product within the oral cavity. In some embodiments, the confectionery product may be used without a stick, by being positioned within the oral cavity.

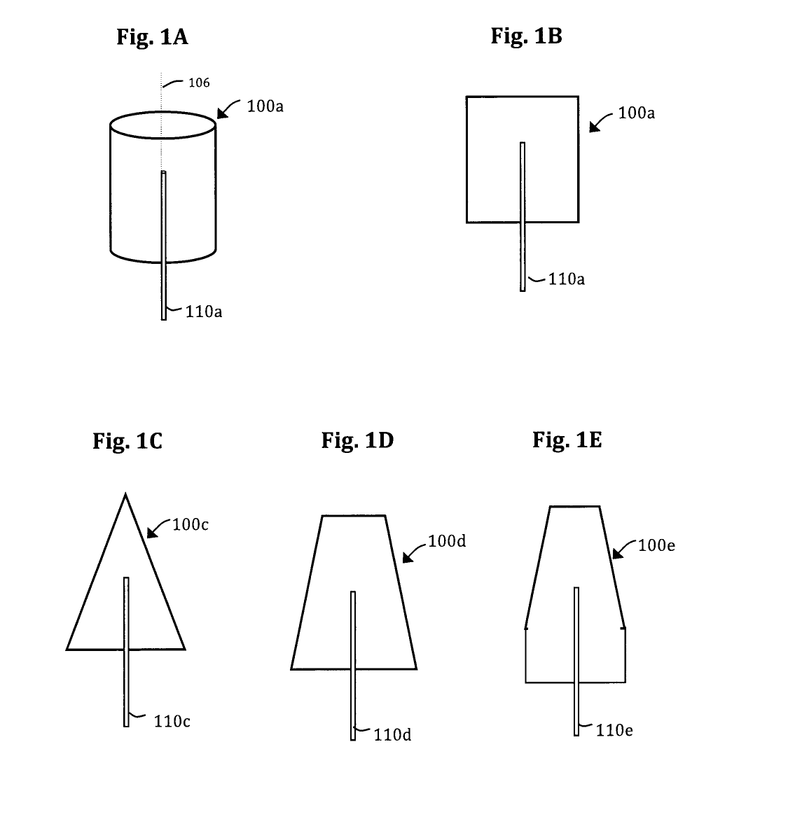

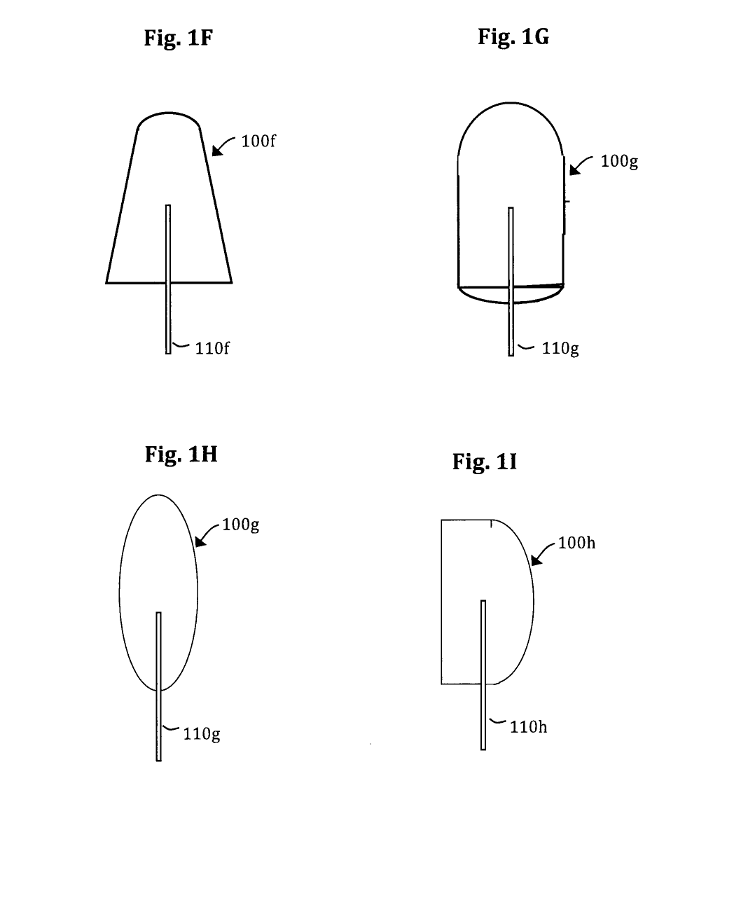

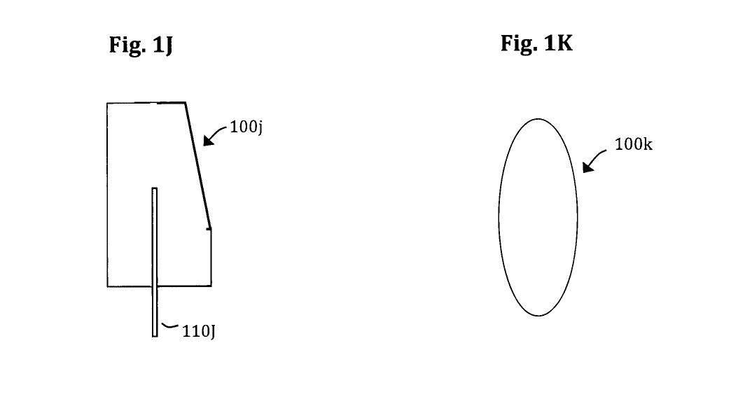

FIGS. 1A-1K schematically illustrates perspective view or cross sectional views of various non limiting shapes of abrasive confectionery products, according to some embodiments. Various particles dispersed within the confectionery products are omitted from FIGS. 1A-1K for clarity purposes only. As shown in FIGS. 1A-J the confectionery product may or may not be symmetrical and may or may not include a stick. FIG. 1A schematically illustrates a perspective view of a confectionery product 100a as cylinder shape like. Confectionary product 100a comprises an elongated stick 110a partially disposed in confectionery product 100a along axis 106.

FIG. 1B schematically illustrates cylindrical candy 100a of FIG. 1A as a cross-section view along the rotational symmetry axis 106.

FIG. 1C schematically illustrates a cross section of a cone shape-like confectionery product 100c, which comprises an elongated stick 110c partially disposed in confectionery product 100c.

FIG. 1D schematically illustrates a cross-section view of a confectionery product 100d formed as a truncated cone shaped, which comprises an elongated stick 110d partially disposed in confectionery product 100d.

FIG. 1E schematically illustrates a cross-section view of a confectionery product 100e formed as a combination of a cylinder and a truncated cone. Confectionary product 100e comprises an elongated stick 110e partially disposed in confectionery product 100e.

FIG. 1F schematically illustrates a cross-section view of a curved polygon-shape like confectionery product 100f, comprising stick 110f partially disposed in confectionery product 100f.

FIG. 1G schematically illustrates a cross-section view of a curved polygon-shape like confectionery product 100g, comprising stick 110g partially disposed in confectionery product 100g.

FIG. 1H schematically illustrates a cross-section view of an ellipsoid-like confectionery product 100h, comprising stick 110h partially disposed in confectionery product 100h.

FIG. 1I schematically illustrates a cross-section view of a non symmetrical confectionery product 100i, comprising stick 110i partially disposed in confectionery product 100i.

FIG. 1J schematically illustrates a cross-section view of yet another non symmetrical confectionery product, 100j, comprising stick 110j, partially disposed in confectionery product 100j.

FIG. 1K schematically illustrates a cross-section view of an ellipsoid-like confectionery product 100k, which does not include a stick.

Typically, without limiting, the diameter of the confectionery product (such as, for example, confectionery product 100a in FIG. 1A, or largest diameter in case of non-cylindrical shapes), is between about 1 cm to about 3 cm. Preferably, the diameter, or smallest diameter in case of tapered shapes such as, for example, 100c, 100d or 100e (FIGS. 1C-E), or the rounded cap (FIGS. 1F-G) is such as to avoid gagging (gag reflex) when the base (dorsal region) of the tongue is abraded or eroded. Typically, without limiting, the length of a confectionery product, such as, for example, confectionery product 100a is between about 3 cm to about 7 cm.

Stick 110 is made of or comprises any suitable material, such as wood, plastic, paper or a combination thereof. In some embodiments, the stick is a safety stick. In some embodiments the stick is a toothpick. The stick may be used to guide, control, manipulate and move the confectionery product within the oral cavity. The use of a stick aids the user in reaching deep regions in the oral cavity, such as, for example, the dorsal region of the tongue, without inducing a gag reflex, as detailed below herein.

In some embodiments of the invention, the confectionery product (such as, for example, confectionery product 100k in FIG. 1K) does not comprise a separate stick, and confectionery product can be turned or rolled using the body (bulk) of confectionery product, for example, within the oral cavity, such as, for example, by aid of the tongue. In other embodiments, the confectionery product is formed with an extension resembling a stick, made of the bulk material or another material to help in guiding/moving the confectionery product within the oral cavity. In some embodiments, the confectionery product and a stick are provided separately (for example, in the same package) and the stick may be pushed and/or locked inside a hollow in confectionery product before use.

### Composition

In some embodiments of the invention, the confectionery product comprises a hard or rigid bulk material (‘bulk’). The bulk comprises or resembles an amorphous or partially crystallized sugar or a solid solution or mixture of crystallized sugar with softer or fluid material, for example, with water having between about 2% to about 10% moisture. In some embodiments, the bulk exhibits hardness of about or higher than grade 10 in Table 10.3 on page 357 of Harry T Lawless and Hildegarde Heymann, Sensory Evaluation of Food, Springer 1999, ISBN 083421752X, 9780834217522).

In some other embodiments of the invention the confectionery product is semi-hard such as toffee or gummy or, for example, having hardens of between about grade 6 and 8 in Table 10.3 cited above. Optionally or additionally, the confectionery product is elastic and/or flexible resembling rubber or polymeric material such as silicone.

According to some embodiments, the confectionery product comprises abrasive particles substantially uniformly dispersed within the bulk. The abrasive particles are structured to at least partially erode and/or abrade and/or remove and/or scrub and/or loosen deposits in the oral cavity, such as, for example, deposits on the tongue and in grooves and folds of the tongue. Typically and preferably, the particles are harder than the bulk, and in some embodiments the particles are harder than the hard bulk described above. In some embodiments of the invention, the particles dissolve or disintegrate in the mouth and/or gastrointestinal tract. Optionally or alternatively, the particles are mixed with the saliva and are swollen with the saliva.

According to some embodiments, the particles may have any geometrical shape and form, such as, for example a symmetric shape (such as, for example, a circle), an asymmetric shape, and the like. In some embodiments, the particles have coarse or prickly or rough surfaces optionally with sharpened or pointed edges. Optionally or alternatively, the particles have smooth surfaces and abrade deposits on the tongue due to the hardness of the composition of the particles relative to the composition of the deposits.

According to some embodiments, due to the shape and size of the particles, the particles are able to provide deep tongue cleaning, as they may penetrate below the film of the tongue and able to penetrate the inner grooves and folds of the tongue. Such deep cleaning provides an enhanced and superior abrading of deposits of the tongue and results in an improved cleaning of the tongue and enhanced reduction of oral malodor as compared to superficial abrading of the tongue surface.

According to some embodiments, the hard part or parts of the particles (relative to the bulk) comprises compositions of a saccharide such as starch, dextrose, cellulose, wax or waxes and/or a derivate or derivatives thereof. Optionally, other compositions are used such as carbonates or polyols. In some embodiments, the hard part comprises one or more crystalline compositions, optionally as a mixture of crystals.

In some embodiments of the invention, the particles are formed as granules. In some embodiments of the inventions, the particles are formed as capsules comprising an inner core and an external shell. In some embodiments, the shell and/or the core comprise one or more ingredients as described above. In some embodiments, the shell is hard and abrasive and the core is softer than the shell. Optionally or alternatively, the shell dissolves or dissipates in the mouth and the core is hard (such as relative to the shell or the bulk) and functional as an abrasive after the shell has dissolved or disintegrated at least partially. In some embodiments of the invention, the particles are formed both as granules and capsules.

According to further embodiments, the particles are soft, such as to allow the gentle scrubbing/erosion of sensitive areas, such as, for example, a sore oral cavity. In some exemplary embodiments, the confectionery product comprises soft particles that may be used to treat a child/infant sore oral cavity, that may be infected by, for example, bacteria, virus or fungi. In some embodiments, the confectionery product may include soft microcapsules containing one or more health enhancing agents (such as, for example, antifungal agent, anti bacterial agent, anti-viral agent and the like), for the treatment of various oral cavity related conditions, such as, for example, Aphtha.

In some embodiments, the particles may include one or more ingredients such as, for example, health enhancing agents, beneficial composition (e.g. propolis), breath or mouth freshener (herein a ‘freshener’, e.g. menthol), fragrance, and the like, or any combination thereof, for delivery locally in the oral cavity and/or systemic delivery.

According to some embodiments, the ingredients in the particles may be used to treat various oral cavity related conditions, such as, for example, candidiasis, bacterial and viral infections, burning mouth syndrome, xerostomia, abscess, and the like.

The health enhancing agent may include, for example, a pharmaceutical active agent, a natural product, or both. The health enhancing agent may be one which has activity in reducing, preventing or treating a disorder of the oral cavity, such as, for example, but not limited to: candidiasis, halitosis, herpes simplex, lichen planus, leukoplakia, stomatitis, burning mouth syndrome (BMS), dry mouth syndrome (xerostomia), mouth ulcers, gum inflammation, and the like. The health enhancing agent may be associated with other indications or beneficial effects. Exemplary health enhancing agents may include such agents as, but not limited to: analgesic agents (such as, for example, paracetamol, non-steroid anti-inflammatory drugs, salicylates, ibuprofen and lidocaine); antibacterial agents (such as, for example, dichlorobenzyl alcohol, amylmetacresol and antibiotics (penicillins carbapenems, cephalosporins aminoglycosides, bacitracin, gramicidin, mupirocin, chloramphenicol, thiamphenicol, lincomycin, clindamycin, macrolides, novobiocin, polymyxins, rifamycins, spectinomycin, tetracyclines, vancomycin, teicoplanin, streptogramins, anti-folate agents, sulfonamides, trimethoprim, pyrimethamine, nitrofurans, methenamine mandelate, methenamine hippurate, nitroimidazoles, quinolones, fluoroquinolones, isoniazid, ethambutol, pyrazinamide, para-aminosalicylic acid, cycloserine, capreomycin, ethionamide, prothionamide, thiacetazone and viomycin)); antiseptics (such as, for example, chlorhexidine and salts thereof, benzalkonium and salts thereof, triclosan and cetylpyridium chloride); cholinergic agents (such as, for example, pilocarpine and cevimeline); antifungal agents (such as, for example, tolnaftate, miconazole, fluconazole, clotrimazole, econazole, ketoconazole, itraconazole, terbinafine, amphotericin, nystatin and natamycin); steroids (such as, for example, prednisone acetate, prednisone valerate, prednisolone, alclometasone dipropionate, fluocinolone acetonide, dexamethasone, methylprednisolone, desonide, pivolate, clocortolone pivolate, triamcinolone acetonide, prednicarbate, fluticasone propionate, flurandrenolide, mometasone furoate, desoximetasone, betamethasone, betamethasone dipropionate, betamethasone valerate, betamethasone propionate, betamethasone benzoate, diflorasone diacetate, fluocinonide, halcinonide, amcinonide, halobetasol propionate, and clobetasol propionate); Nutritional supplements (such as, for example, vitamins (for example, vitamin A, those in the vitamin B family, vitamin C, those in the vitamin D family, vitamin E and vitamin K), minerals, amino acids; Ethereal oils (such as, for example, those derived from mint, sage, fir, lavender, basil, lemon, juniper, rosemary, eucalyptus, marigold, chamomile, orange, and the like); Herbal and plant products (such as, for example, kava kava (Piper methysticum), purple coneflower (Echinacea sp.), horsetail (Equisetum arvense), myrrh (Commiphora myrrha), agrimony (Agrimonia sp.), cat's claw (Uncaria tomentosa), thuja (Thuja sp.), propolis, poke root (Phytolaccae radix), elecampane (Inula helenium) and goldenseal (Hydrastis canadensis)).

In some embodiments, the ingredients may include an active compound or a combination of active compounds able to react or combine with materials in the deposits on the tongue. For example, compositions of divalent metal such as zinc or copper that react with the sulfur compounds to reduce the malodor.

In some embodiments, the particles may be formed and/or adapted for a slow release in the oral cavity and/or gastrointestinal tract of composition or compositions comprised and/or contained in the particles. Herein the term ‘slow release’ is as used in the art.

In some embodiments, the release of ingredients from the particles may be controlled such that the release is conditioned to occur under specific conditions, such as, for example, at a specific temperature range, specific pH range, under specific mechanical force, and the like. For example, at pH=8, the release may occur in the oral cavity. For example, at pH=2, the release may occur in the stomach. This way, the spatial-temporal release of the ingredients may be controlled such that the ingredients are released in a designated site of action (such as, for example, in the gastrointestinal tract) at required timing.

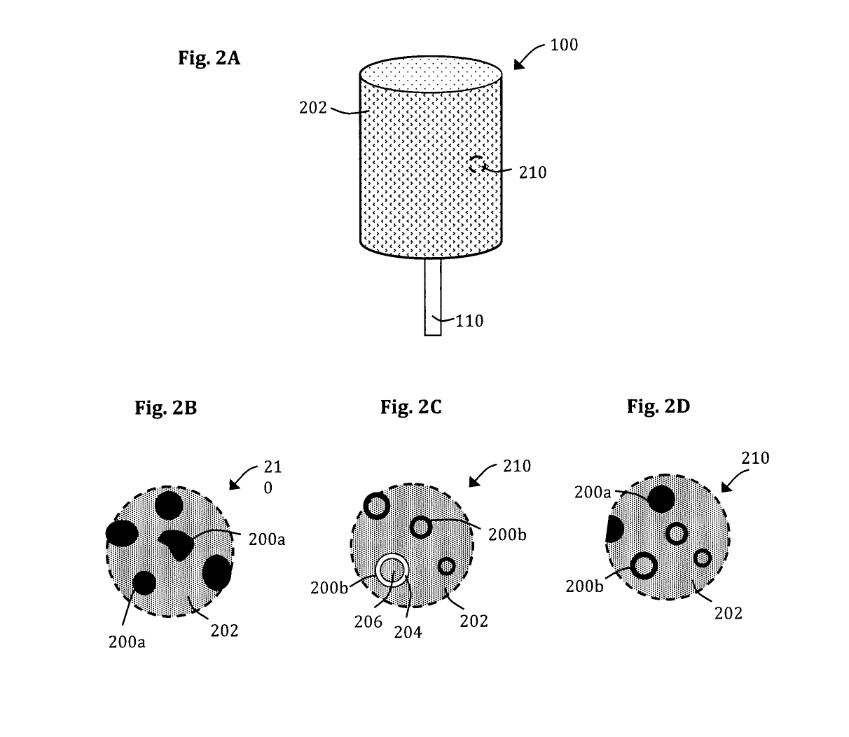

FIG. 2A schematically illustrates a perspective view of abrasive confectionery product 100 comprising a bulk 202 with substantially uniformly dispersed abrasive particles 200 (shown schematically as spots), according to exemplary embodiments of the invention.

In some embodiments, particles 200 comprise between about 0.5% and about 5% by weight of confectionery product 100 such as to provide at least some extent of abrading and/or eroding and/or scraping of the surface of the tongue while avoiding or diminishing irritation or pain or damage to the tongue. In some typical embodiments, without limiting, particles 200 comprise about 2% of the weight of the confectionery product (disregarding the weight of stick 110).

In some embodiments, the uniform dispersion is considered wholly or at least within some volume, such as about 10% of the volume of confectionery product 100. Optionally, the uniform dispersion is approximately uniform, such as with deviations of about 10% between regions of the volume.

A sample 210 of bulk 202 with particles 200 is indicated in FIG. 2A and schematically enlarged in FIGS. 2B-D.

FIG. 2B schematically illustrates an enlarged view of sample 210 of confectionery product 100 comprising bulk 202 with abrasive particles as granules 200a.

FIG. 2C schematically illustrates an enlarged view of sample 210 of confectionery product 100 comprising bulk 202 with abrasive particles as capsules 200b comprising an external shell 204 and core 206.

FIG. 2D schematically illustrates an enlarged view of sample 210 of confectionery product 100 comprising bulk 202 with abrasive particles as a mixture of granules 200a and capsules 200b.

In some embodiments of the invention, the sizes of particles 200 (e.g. represented by maximal or average diameter) fall in some non-limiting range between about 50 μm and about 1 mm. In some embodiments, the percentage of particles or sizes nearer to the average or median size is larger than the percentage nearer the extreme sizes. For example, in a certain population of particles 200 in confectionery product 100 with a size distribution between about 50 μm to about 850 μm, the range between 50 μm to about 250 μm and the range between 650 μm to about 850 μm is about 10% of the population each, and each of the ranges between 250 μm to about 450 μm and the range between 450 μm to about 650 μm is about 40% of the population.

In some embodiments, the size distribution of the particle sizes within the abrasive product may vary. For example, particles in the range of about 50-850 micron may be distributed as follows: 20-80% may have a size in the range of about 50-350 micron; 20-80% may have a size in the range of about 350-650 micron; and 20-80% have a size in the range of about 650-850 micron. For example, a size distribution of particles in the range of about 50-850 micron may be: about 40% may have a size in the range of about 50-350 micron; about 40% may have a size in the range of about 350-650 micron and about 20% may be in the range of 650-850 micron.

In some embodiments, the density of the particles is in the range of 0.1-2.0 gr/ml. In some embodiments, the density of the particles is in the range of 0.1-1.0 gr/ml. For example, the density may be in the range of about 0.5-2 gr/ml. Such density/concentration enhances the abrasive quality of the confectionery product, as it allows more abrasive particles to contact larger area of surfaces in the oral cavity. Hence, such concentration/density of the abrasive particles within the bulk material allows an overall enhanced oral malodor diminishing/removing effect over time.

According to further embodiments, using confectionery product 100 with various sizes of particles 200 (e.g. as described above) enable abrading various textures and/or characters and/or hardness of the deposits in the oral cavity in general and on the tongue in particular. In addition, the various structures of the particles enables deep tongue cleaning such that the inner grooves of the tongue are abraded by the particles that are able to penetrate and reach those grooves.

According to other embodiments of the invention, the size distribution and/or concentration/density of dispersed particles may vary throughout or between different parts of the confectionery product, wherein within each region the distribution is substantially uniform.

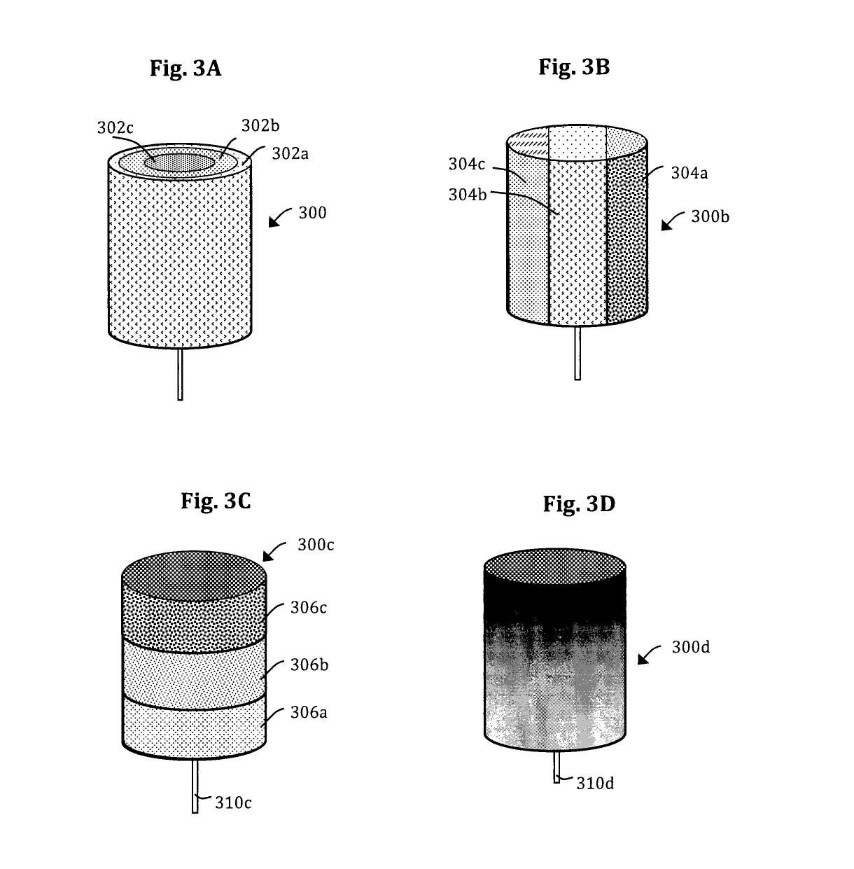

FIG. 3A schematically illustrates a perspective view of abrasive confectionery product 300 having dispersed abrasive particles (schematically shown as spots) in concentric layers or rings 302a-302c, each with different concentration/density schematically shown as different levels of rendering. It should be noted that instead of and/or in addition to the different concentrations, in some embodiments the rings have different composition or size distributions of the particles (such as, particles 200 in FIG. 2). For example, outermost ring 302a has an average size of 200 μm in one standard deviation, innermost ring 302c has an average size of 600 μm in one standard deviation and intermediate ring 302b has an average size of 400 μm in one standard deviation. It should be emphasized that the three rings are provided as an example only and any number of rings may be constructed. In some embodiments, instead of discrete separate rings such as rings 302a-302c, the concentration/density and/or size distribution, at least approximately, vary continuously in a concentric manner (see also FIG. 3D for continuous variation).

FIG. 3B schematically illustrates a perspective view of abrasive confectionery product 300b having dispersed abrasive particles (schematically shown as spots) in layered regions 304a-304c parallel to a symmetry axis of the confectionery product, each with different concentration/density schematically shown as different levels of rendering. It should be noted that instead of and/or in addition to the different concentrations/densities, in some embodiments the regions have different composition or size distributions of particles. For example, right region 304a (with respect to the drawing) has an average size of 200 μm in one standard deviation, left region 304c has an average size of 600 μm in one standard deviation and intermediate region 304b has an average size of 400 μm in one standard deviation. It should be emphasized that the three regions are provided as an example only and any number of regions may be constructed. In some embodiments, instead of discrete separate regions such as regions 304a-304c, the concentration/density and/or size distribution, at least approximately, vary continuously in a layered manner parallel to a symmetry axis (see also FIG. 3D for continuous variation).

FIG. 3C schematically illustrates a perspective view of abrasive confectionery product 300c having dispersed abrasive particles (schematically shown as spots) in layered regions 306a-306c perpendicular to a symmetry axis, each with different concentration/density schematically shown as different levels of rendering. It should be noted that instead of and/or in addition to the different concentrations, in some embodiments the regions have different composition or size distributions of particles. For example, bottom region 306a (nearest to stick 310c) has an average size of 200 μm in one standard deviation, intermediate region 306b has an average size of 400 μm in one standard deviation and topmost region 306c has an average size of 600 μm in one standard deviation. It should be emphasized that the three regions are provided as an example only and any number of regions may be constructed. In some embodiments, instead of discrete separate regions such as regions 306a-306c, the concentration and/or size distribution, at least approximately, vary continuously in a layered manner parallel to a symmetry axis as schematically illustrated in FIG. 3D.

FIG. 3D schematically illustrates a perspective view of abrasive confectionery product 300d having dispersed abrasive particles in continuously varying concentrations (schematically shown as varying levels of rendering) in layered regions perpendicular to the symmetry axis of the confectionery product, where the lower part (nearest to stick 310d) having a lower concentration relative to the upper parts and wherein the top part has the highest concentration. It should be noted that instead of and/or in addition to the different concentrations, in some embodiments the regions have varying different composition or size distributions of particles.

According to some embodiments, the abrasive confectionery product may further include any type of additives, food coloring, coatings, and the like, that may be an integral part of the product, and/or may coat the product.

Some exemplary compositions (approximate fractions by weight) are listed below.

Bulk: 75% isomalt, 25% water, 0.25% citric acid, 0.025% sucralose

Particles, as capsules: 2%,

Variant i: capsules with an inert substance, Tricalcium Phosphate (TCP)

Variant ii: capsules with Propolis

Variant iii: capsules with Zinc-Gluconate and inert substance (TCP) at a 1:3 respective ratio

Variant iv: capsules with Zinc, Propolis and inert substance (TCP) at a 1:4:3 respective ratio.

In some embodiments, in order to avoid exceeding the recommended daily allowance of ingredients within the particles, some particles may include inert materials. For example, for Zinc (recommended daily allowance of 15 mg for an adult person), the expected number of confectionery products consumed daily should be considered. Therefore, confectionery products with zinc compounds as exemplified above also comprise capsules with inert material (such as, for example, TCP). The use of Zinc with an inert material (such as TCP) may further provide additional advantage by improving the tolerability of the product by a user, by improving the taste of the confectionery product by reducing or eliminating the taste of the Zinc.

According to some embodiments, the confectionery product is consumable, such that it is consumed as it is being used. During consumption of the product (for example, by dissolvation in the oral cavity, by softening of the product by saliva, and the like), the abrasive particles are exposed to surfaces of the oral cavity and able to abrade those surfaces. The exposure rate of the abrasive particles is constant, such that the abrasive quality and ability of the confectionery product is substantially constant throughout the consumption of the product. That is, at any time point after consumption of the product, the abrasive quality does not significantly change, meaning that the abrasive quality at time point zero (before being used) is substantially equal to the abrasive quality at various time points after consumption. This is advantageous since the abrading quality, conferred by the abrasive particles does not significantly diminish over time and hence the product provides longer lasting beneficial effect on oral malodor and other oral cavity related condition.

The abrasive quality of a confectionery product may be determined, for example, by the pull out test. A pull-out test may be used to quantitatively determine the abrasive qualities of edible compositions by measuring the amount of interface resistance when the edible composition is removed from a standard displacement medium. The force required to remove the confectionery object and/or the lateral stress on the displacement medium can be can be measured in kN (kilo Newtons), kPa (kilo Pascals) and/or in grams/cm². The abrasive particles should provide sufficient friction to effectively abrade the oral cavities, but should not be excessively abrasive so as to cause damage, injury, discomfort or pain to the user. In some embodiments, the abrasive quality is in the range of about 10-25 grams/cm². In some embodiments, the abrasive quality is in the range of about 13-18 grams/cm². In some embodiments, the abrasive quality is in the range of about 14-17 grams/cm². Examples 1 and 2 herein below detail an exemplary abrasive test using the confectionery product of the invention.

### Manufacturing

Confectionary product 100 can be manufactured by any method known in the art or a variation and/or combination thereof. For example, sugar and/or a substitute thereof is boiled in a solvent such as water to form syrup which is cooled in molds or formed into required shape in a batch rolling machine.

Particles 200 are formed by methods such as microspheres fabrication, for example, polymerization in a stirred emulsion or suspension, and mixed with the syrup of confectionery product 100 before the syrup cools.

For some microencapsulation techniques, see, for example, http://www.karmat.com, or Simon Benita, Microencapsulation: Methods and Industrial Applications, CRC Press, 2006, 2nd edition, ISBN 0824723171, 9780824723170.

A manufacturing non-limiting partial example:
i. Heat about 300 gr Isomalt with about 100 gr of water to boil at about 165 C.°
ii: stop heating
iii. mix all other ingredients separately
iv: Add other ingredients (e.g. particles, citric acid, sucralose, coloring, flavors) to Isomalt at about 140 C.°
v. Mix until the other ingredients are evenly dispersed
vi. Pour into mold
vii. Insert stick
viii. Cool to warm touch
ix. Release from mold

In case of non-uniform concentration and/or density and/or size distribution of particles 200, the molding may be carried out in steps in the same or different molds, each with different composition of particles 200.

### Use

According to some embodiments, the confectionery product may be used to abrade, scrape, erode, release and/or loosen deposits in the oral cavity, such as, for example, on the tongue surface and in grooves and folds of the tongue. As detailed above, by abrading the various deposits in the oral cavity, oral malodor is reduced or diminished. The abrading action may result from a reciprocal movement in any direction of the confectionery product in the oral cavity, which in turn facilitates said scraping, abrading and/or loosening of the deposits (such as food debris, microorganisms and metabolic products), which may be classified as the source of the oral malodor and other oral cavity related conditions. The dislodged substances may then be swallowed by the user as the confectionery product is consumed. The abrading action may be performed by various ways, actively or passively, such as, for example, manually maneuvering the confectionery product with the aid of a stick associated with the confectionery product (or an integral protrusion of the product itself, as detailed above); by maneuvering the confectionery product in the oral cavity, by using the tongue and/or the lips; passively, by positing the confectionery product within the oral cavity, and dissolving it with various regions of the oral cavity (such as the tongue, palate, and the like), or any combination thereof.

According to some embodiments, the confectionery product does not induce a gag reflex, even when used for deep tongue cleaning. The pharyngeal reflex or gag reflex is a reflex contraction of the back of the throat evoked by touching the soft palate. It prevents objects from entering the throat except as part of normal swallowing and helps prevent choking. Different people have different sensitivities to the gag reflex. Swallowing unusually large objects or placing objects in the back of the mouth, like a toothbrush, may cause pharyngeal reflex. A gagging severity index (GSI) related to dental treatments (Dickinson et. al. (2000)) is represented in Example 3. Many treatments for oral malodor are unable to treat the dorsoposterior portion of the tongue without stimulating the gag reflex. A product that can be manipulated such that it does not stimulate the gag reflex enables a more efficient and effective treatment of oral malodor and other oral cavity related conditions, as more surfaces of the oral cavity can be abraded to remove debris, microorganisms and other factors that cause oral malodor and other oral cavity related conditions. Example 3 demonstrates a gag reflex test using the confectionery product of the invention.

In some embodiments of the invention the confectionery product is flexible, at least to some degree and at least one or more of parts thereof, such as the upper part. With the confectionery product having a flexible structure, the stick can be manipulated to maneuver confectionery product in the oral cavity, applying medication and/or or breath or mouth freshener and/or active compounds (e.g. divalent metal compounds) or other compositions and optionally or additionally, abrading deposits such as plaque off the gums or teeth.

In some embodiments, the stick associated with the confectionery product is formed such as to allow moving and guiding the confectionery product on the tongue by moving stick with the tongue itself. For example, the stick may be at least partially flattened, allowing holding stick with the teeth and/or lips while the tongue moves stick. The use of a stick may allow, for example, to selectively position the confectionery product on the pharyngeal part of the tongue, which faces the oropharynx, in order to effect an abrading action thereon, without inducing the gag reflex normally caused by contact with the pharynx.

In some embodiments of the invention, the confectionery product may provide extended functionality as a mouth hygiene tool by forming one or two ends of stick into a sharpened tapered shape that can be used as an integral toothpick, where optionally the end of the stick inside confectionery product provides a toothpick after the confectionery product is consumed.

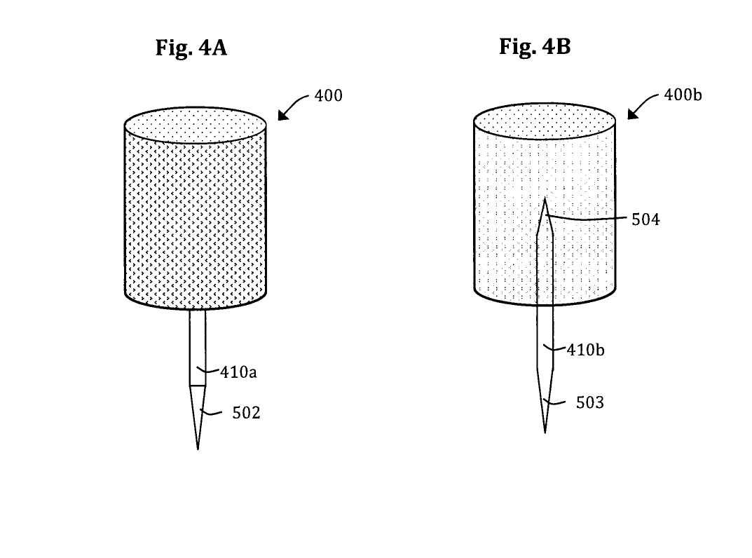

FIG. 4A schematically illustrates a perspective view of abrasive confectionery product 400 having stick 410a with sharpened end 502 outside confectionery product 400. FIG. 4B schematically illustrates perspective view of abrasive confectionery product 400b having stick 410b with sharpened ends 503 and 504 outside and inside confectionery product 400b, respectively, according to exemplary embodiments of the invention. The toothpick ends 502, 503 and/or 504 of the stick can be used, for example, to remove food residues and/or plaque and/or deposits on or between the teeth.

In some embodiments, the stick comprises or formed with bumps and/or pits or otherwise has a rough surface that can be used in abrading and/or eroding the deposits on the tongue further or differently to confectionery product.

In some embodiments, the stick has perforations or dents (or other structures) allowing to clip or trim the stick to a convenient length for abrading or eroding the tongue. In some embodiments, the stick is a safety stick. In some embodiments, the stick is a toothpick. In some embodiments, the stick is flexible.

In some embodiments of the invention, when confectionery products such as illustrated in FIGS. 3C-D are used, the higher concentration/density and/or suitable sizes of particles at the upper side of confectionery products (relative to end with the stick) provide enhanced abrasion in the base of the tongue (posterior dorsal part) where the malodor producing deposits are likely to be found, relative to other parts of the tongue.

As detailed herein, the use of the confectionery product of the present invention results in improved reduction up to complete elimination of oral malodor. Several methods are known in the art to assess oral malodor. For example, organoleptic scoring is known in the art as a method of assessing the severity of oral malodor based on the sensitivity of the human olfactory sensory system. Example 4 below demonstrates an organoleptic scoring test for using the confectionery product of the invention.

Another known method of assessing oral malodor is by using Halimeter. Halimeters are portable sulfide meters that detect the volatile sulfur compounds in oral malodor, measured in parts per billion (ppb) of volatile sulfur compounds. The main component of the volatile sulfur compounds, responsible for oral malodor is methylmercaptan and Halimeters are most sensitive to Hydrogen Sulfide (H₂S), but as there is good correlation between the two, the Halimeter is still useful for detecting oral malodor. Studies have shown that there is significant correlation between Organoleptic scoring and Halimeter readings (Winkel and Tangerman, 2005; Baharvand et al., 2008). Halimeters are portable and easy to use but the presence of certain chemicals found in perfume, deodorants and crucially, mouthwashes can disrupt Halimeter readings. Halimeter reading tests using the confectionery product of the invention is detailed hereinbelow in Examples 5 and 6.

### General

The following non-limiting characterizations of terms are applicable in the specification and claim unless otherwise specified or indicated in or evidently implied by the context, and wherein a term denotes also variations, derivatives, inflections and conjugates thereof.

The terms ‘about’, ‘close’, ‘approximate’, and ‘practically’ denote a respective relation or measure or amount or quantity or degree yielding an effect that has no adverse consequence or effect relative to the referenced term or embodiment or operation or the scope of the invention.

The terms ‘similar’, ‘resemble’, ‘like’ and the suffix ‘-like’ denote shapes and/or structures and/or operations that look or proceed as, or approximately as the referenced object.

The terms ‘constant’, ‘uniform’, ‘continuous’ and other seemingly definite terms denote also close or approximate respective terms.

The terms ‘vertical’, ‘perpendicular’, ‘parallel’, ‘opposite’, ‘straight’ and other angular and geometrical relationships denote also approximate yet functional and/or practical, respective relationships.

The terms ‘preferred’, ‘preferably’, ‘typical’ or ‘typically’ do not limit the scope of the invention or embodiments thereof.

The terms ‘comprises’, ‘comprising’, ‘includes’, ‘including’, ‘having’ and their inflections and conjugates denote ‘including but not limited to’.

The term ‘may’ denotes an option which is either or not included and/or used and/or implemented, yet the option comprises a part of the invention.

Unless the context indicates otherwise, referring to an object in the singular form (e.g. “a thing” or “the thing”) does not preclude the plural form (e.g. “the things”).

The present invention has been described using descriptions of embodiments thereof that are provided by way of example and are not intended to limit the scope of the invention or to preclude other embodiments. The described embodiments comprise various features, not all of which are necessarily required in all embodiments of the invention. Some embodiments of the invention utilize only some of the features or possible combinations of the features. Alternatively and additionally, portions of the invention described or depicted as a single unit may reside in two or more separate entities that act in concert or otherwise to perform the described or depicted function. Alternatively and additionally, portions of the invention described or depicted as two or more separate physical entities may be integrated into a single entity to perform the described/depicted function. Variations related to one or more embodiments may be combined in all possible combinations with other embodiments.

When a range of values is recited, it is merely for convenience or brevity and includes all the possible sub-ranges as well as individual numerical values within that range. Any numeric value, unless otherwise specified, includes also practical close values enabling an embodiment or a method, and integral values do not exclude fractional values. A sub-range values and practical close values should be considered as specifically disclosed valued.

Terms in the claims that follow should be interpreted, without limiting, as characterized or described in the specification.

In order that this invention may be better understood, the following examples are set forth. These examples are for the purpose of illustration only and are not to be construed as limiting the scope of the invention in any manner.

### EXAMPLES

### Example 1

### Abrasive Quality—Measurements of Abrasiveness of a Confectionery Product

A test designed to measure the abrasiveness of a confectionery product is a pullout test, which can quantitatively determine the abrasive qualities of edible compositions by measuring the amount of interface resistance when the edible composition is removed from a standard displacement medium.

In the test, cubical structures (approximate size of 3 cm×2.2 cm×7.7 cm) of the confectionery product of the invention were prepared (Composition: 98% w/w isomalt and 2% w/w particles, in the form of microcapsules (size distribution of about 50-850 micron, of which about 40% are in the range 50-350 micron, about 40% in the range of 350-650 micron and about 20% in the range of 650-850 micron), uniformly distributed within the bulk material). On a smooth, horizontal surface, a delicate sandpaper in the length of 1 meter was taped (CAMI 120, FEPA P120, grain size 110 micron). To the cubical confectionery product a hook was attached to which a force meter was attached. The confectionery product was placed (under different conditions) on the sandpaper and was pulled along a distance of 40 cm, while measuring the force during movement, with force meter.

### Example 2

### Abrasive Quality Over Time

Measurements of the abrasiveness of a confectionery product over time were preformed essentially as above. In order to simulate consumption of the confectionery product, the product is dissolved for a predetermined time (1 minute) at predetermined temperature (37° C.) in a predetermined medium (water). Various experiments may be performed using different parameters, such as, longer/shorter dissolvation rates, higher/lower temperatures, and/or various mediums (such as, for example, saliva, higher/lower viscosity liquid medium). After each dissolvation, at various time points, the abrasive quality was measured. In order to normalize results, at each measurement of the abrasive quality, weights were used in order to maintaining equal weight for the force measurements. Likewise, the size of the abrasive surface are (measured in cm²) was taken into the calculation. The results, which are presented in Table 1 below, demonstrate that the abrasive quality of the product (as measured in grams per surface area), remains substantially constant at various time points after its dissolvation.

TABLE 1

| | Dissolvation | Surface | Force (gr) | Abrasive |
|---|---|---|---|---|
| Weight | Time | area | during | quality |
| (gr) | (minutes) | (cm²) | movement | (gr/cm²) |
| 91.8 | 2 | 19.1 | 330 | 17.4 |
| 80.6 | 2 | 17.76 | 250 | 14.07 |
| 74.7 | 2 | 15.5 | 220 | 14.19 |

### Example 3

### Gag Reflex and Tolerability

The following is a gag reflex test protocol used to assess the gag reflex when using confectionery products. Table 2 herein below, lists an exemplary Gagging severity index (adapted from Dickinson, 2000), at various conditions.

TABLE 2
Gagging severity index (GSI)

| Severity level | Gagging reflex is: |
|---|---|
| I | Very mild, occasional and controlled by the patient |
| II. | Mild, and control is required by the patient with |
| | reassurance from the dental team. |
| III. | Moderate, consistent and limits treatment options |
| IV. | Sever and treatment is impossible |
| V. | Very sever; affecting patient behavior and dental |
| | attendance and making treatment impossible. |

A total of 75 subjects were enrolled to the study. Tolerability of the abrasive confectionery product was captured by reporting on nausea and itching sensation by users. In this study the, abrasive confectionery product was well tolerated in 85% of the cases, 12% reported on minimal nausea and 1 cases reported on severe nausea with no need for any treatment.

### Example 4

### Organoleptic Scoring

The following is an Organoleptic testing protocol used to assess oral malodor at various conditions. Subjects remain quiet and maintain a closed mouth for a period of 30 sec. Subjects are then requested to exhale through the mouth with moderate force into a sampling bag for 2 to 3 sec to prevent the dilution of odor with lung and room air. This procedure is repeated tow or more times. Three malodor graders, trained to perform the examination, estimate the odor at a distance of approximately 10 cm from the sampling bag. The scores are then estimated on a scale of 0 to 5, and the mean values of the three graders are used. Table 3 below shows an organoleptic scoring scale and criteria.

TABLE 3
organoleptic scoring scale and criteria.

| SCALE | CATEGORY | DESCRIPTION |
|---|---|---|
| 0 | Absence of odor | Odor cannot be detected |
| 1 | Questionable odor | Odor detectable, although the examiner |
| | | could not recognize it as malodor |
| 2 | Slight malodor | Odor deemed to exceed the threshold |
| | | of malodor recognition |
| 3 | Moderate malodor | Malodor is definitely detected |
| 4 | Strong malodor | Strong malodor is detected, but can be |
| | | tolerated by examiner |
| 5 | Severe malodor | Overwhelming malodor is detected and |
| | | cannot be tolerated by examiner |
| | | (examiner instinctively averts nose) |

A total of 75 subjects were enrolled to the study. Subjects who have met the selection criteria, and have signed the informed consent were randomly divided into 5 study groups. The study is a double blind randomized controlled comparative study. Both the subjects participating in the study and the investigators are blinded to the kind of product used for the treatment. Efficacy endpoint is organoleptic assessment at baseline and 4 consecutive visits at 10, 60 and 150 minutes.

Each experimental group (15 participants) tested a different confectionery product:
G1: Control group—This group was treated by lollipop Candy, without any abrasive capabilities and without any additional ingredients. (Composition of the confectionery product: Isomalt 300 gr, Water 100 gr, sucralose 0.1 gr, citric acid 1 gr).
G2: Abrasive confectionery product: This group was treated by an abrasive confectionery product having abrasive particles, according to embodiments of the present invention. (Composition of the confectionery product: Isomalt 300 gr, Water 100 gr, sucralose 0.1 gr, citric acid 1 gr, 2% w/w of particles in the form of microcapsules (ranging in size from about 50 to 850 microns), containing 6 gr of TCP (Tricalcium Phosphate)).
G3: Abrasive confectionery product wherein the particles include propolis, according to embodiments of the present invention. (Composition of the confectionery product: Isomalt 300 gr, Water 100 gr, sucralose 0.1 gr, citric acid 1 gr, 2% w/w of particles in the form of microcapsules (ranging in size from about 50 to 850 microns), containing 6 gr of propolis).
G4: Abrasive confectionery product wherein the particles include Zinc, according to embodiments of the present invention. (Composition of the confectionery product: Isomalt 300 gr, Water 100 gr, sucralose 0.1 gr, citric acid 1 gr, 1.5% w/w of particles in the form of microcapsules (ranging in size from about 50 to 850 microns), containing 4.5 gr of TCP; and 0.5% w/w of particles in the form of microcapsules (ranging in size from about 50 to 850 microns), containing 1.5 gr of Zinc).
G5: Abrasive confectionery product wherein the particles include Zinc and propolis, according to embodiments of the present invention. (Composition of the confectionery product: Isomalt 300 gr, Water 100 gr, sucralose 0.1 gr, citric acid 1 gr, 0.75% w/w of particles in the form of microcapsules (ranging in size from about 50 to 850 microns), containing 3 gr of TCP; 0.25% w/w of particles in the form of microcapsules (ranging in size from about 50 to 850 microns), containing 0.75 gr of Zinc; and 1% w/w of particles in the form of microcapsules (ranging in size from about 50 to 850 microns), containing 3 gr of propolis).

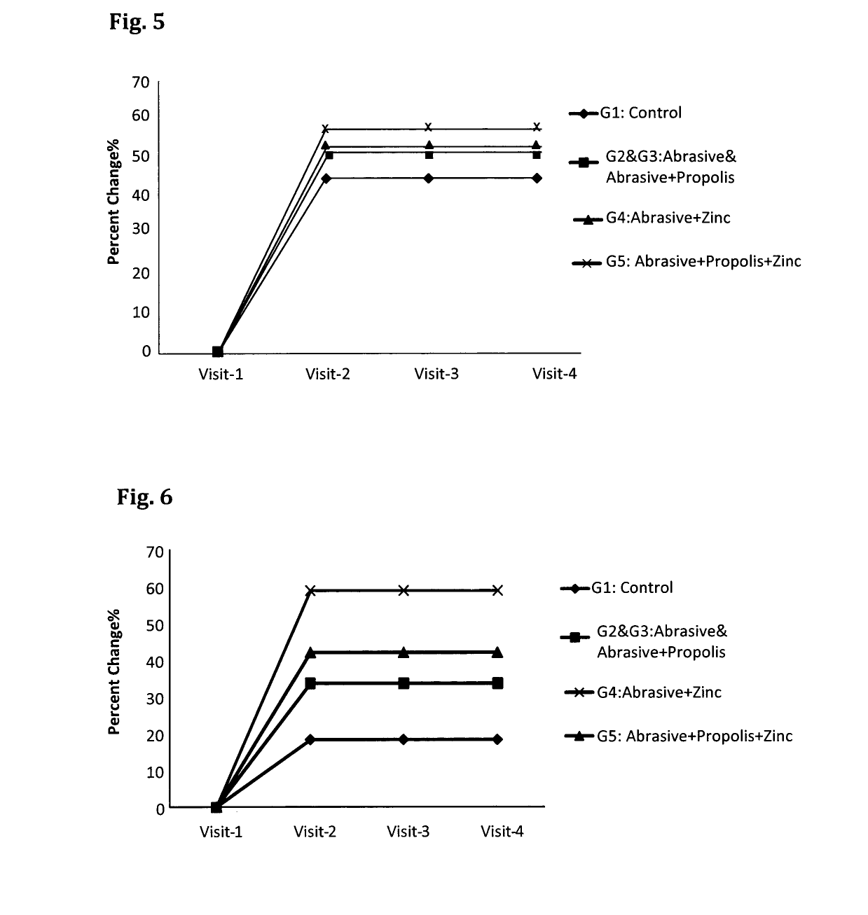

The results are presented in the graph in FIG. 5, as a percent reduction of odor (as determined by the graders), from baseline over time, corrected for the Baseline. As shown in the graph of FIG. 5, whereas in the control (G1) less than 45% reduction in malodor is observed, the use of the abrasive product (groups G2-G5) caused up to 60% reduction in oral malodor.

### Example 5

### Halimetric Readings

The following is an halimetric reading protocol used to assess oral malodor at various conditions. The subject tested should have followed appropriate pre-visit instructions (such as no mouthwash, perfume etc) and the Halimeter should be kept plugged in as preparations are made, as the electrochemical sensor is less accurate for the first 30 minutes after plugged in. The subject is seated sideways on a chair with the arm of the chair raised or otherwise retracted. The subject is facing the front of the Halimeter. The operator is positioned to view and manipulate the adjustments on both the penwriter and the Halimeter. The subject is instructed to close his/her mouth and keep his/her lips sealed in preparation for sampling mouth air. A timer is set for 3 minutes. During these 3 minutes the operator should perform the tasks necessary to prepare the mouth air sampling straw and sampling bag. The short end of the mouth air sampling straw is inserted into the inlet of the Halimeter and the penwriter is switched on and the INPUT set to 0.5 V, CHART SPEED to 2 cm/min, and ZERO to STBY and the pen is lowered to contact the paper. The ZERO knob of the penwriter is adjusted such that the pen falls exactly upon the far right centimeter line on the chart paper. This line represents zero parts per billion (ppb) of volatile sulfur compounds. The free end of the mouth sample straw is directed away from the operator and the subject and the penwriter switched into the record mode (switch from STBY to REC), the ZERO control on the Halimeter is adjusted until the pen scribes a straight line over the far right line on the chart paper (the zero line), such that the Halimeter and recorder are zeroed to ambient air and the reading is “000”±5 ppb. The Halimeter and penwriter should be zeroed prior to each reading.

With the penwriter on and recording, the mouth sample straw is directed towards the subject, during the course of these preparations the subject continues to breathe through the nose and to keep the mouth shut for the 3 minute incubation. When the 3 minutes have passed the subject takes a deep breath in through the nose and with the breath held should lean forward and seal his lips around the wax bead around the straw. The subject hold its breath for the approximately 10-15 seconds it takes to make the recording. The open end of the intact straw should be located in the center of the mouth. The subject should avoid touching the open end of the straw with the lips, tongue, or cheeks. A peak reading is obtained within 2-5 seconds. After about 10-15 seconds, the patient is instructed to pull away from the straw. The recorder pen should return to the “zero” line before making the next recording. For maximum accuracy, incubation and Halimeter sampling the reading should be performed three times, and the average peak reading is calculated.

A total of 75 subjects were enrolled to the study. Subjects who have met the selection criteria, and have signed the informed consent were randomly divided into 5 study groups. The study is a double blind randomized controlled comparative study. Both the subjects participating in the study and the investigators are blinded to the kind of product used for the treatment. Efficacy endpoint is Halimeter readings of particles per billion (ppb) at baseline and 4 consecutive visits at 10, 60 and 150 minutes.

Each experimental group (15 participants) tested a different confectionery product:
G1: Control group—This group was treated by commercial lollipop Candy, without any abrasive capabilities and without any additional ingredients. (Composition as in Example 4).
G2: Abrasive confectionery product: This group was treated by an abrasive confectionery product having abrasive particles, according to embodiments of the present invention (Composition as in Example 4).
G3: Abrasive confectionery product wherein the particles include propolis, according to embodiments of the present invention (Composition as in Example 4).
G4: Abrasive confectionery product wherein the particles include Zinc, according to embodiments of the present invention (Composition as in Example 4).
G5: Abrasive confectionery product wherein the particles include Zinc and propolis, according to embodiments of the present invention (Composition as in Example 4).

The results are presented in the graph shown in FIG. 6, as a percent reduction of odor (as determined by Halimeter readings), from baseline over time, corrected for the Baseline. As shown in the graph of FIG. 6, whereas in the control (G1) less than 20% reduction in malodor is observed, the use of the abrasive product (groups G2-G5) caused up to 60% reduction in oral malodor.

### Example 6

### Halimetric Analysis—Use of an Abrasive Confectionery Product

An additional Halimeter analysis tested the use of the abrasive confectionery product. The product was used to abrade deposits on either the middle (proximal) part of the tongue or on the third (dorsoposterior) part of the tongue. The results show that using the abrasive confectionery product in the deep dorsal end of the tongue was better as compared to using the product on other regions of the tongue. The results are statistically significant and are associated with an average decrease of 16.3%±8.3% of malodor (p=0.054).

The invention claimed is:

1. A consumable abrasive confectionery product adapted to reduce oral malodor comprising substantially uniformly dispersed abrasive particles configured to at least partially erode, scrub, loosen and/or abrade deposits in the oral cavity, wherein the abrasive quality of the confectionery product is substantially constant as the confectionery product is consumed.

2. The confectionery product according to claim 1, wherein the abrasive quality is in the range of about 10-20 gr/cm².

3. The confectionery product according to claim 1, wherein the abrasive quality is in the range of about 13-18 gr/cm².

4. The confectionery product according to claim 1, wherein the density of the particles is in the range of about 0.1-2 gr/ml.

5. The confectionery product according to claim 1, wherein the confectionery product further comprises a stick partially disposed in the confectionery product, wherein the stick is a safety stick, a tooth pick, or both.

6. The confectionery product according to claim 1, wherein the oral cavity comprises tongue, cheek, palate, teeth, gum, and combinations thereof.

7. The confectionery product according to claim 6 wherein the particles are structured to at least partially erode, scrub, loosen and/or abrade deposits on the inner grooves and/or on dorsal region of the tongue.

8. The confectionery product according to claim 1, wherein the particles are selected from granules, capsules, or both.

9. The confectionery product according to claim 8, wherein the capsules comprise an external shell and an internal core.

10. The confectionery product according to claim 8, wherein the capsules comprise ingredients comprising at least one of an inert substance, a medication, beneficial composition, freshener, fragrance or an active compound for reacting with materials in deposits in the oral cavity.

11. The confectionery product according to claim 10, wherein the active compound comprises a compound of a divalent metal.

12. The confectionery product according to claim 11, wherein the divalent metal is Zinc.

13. The confectionery product according to claim 10, wherein the inert substance is Tricalcium Phosphate (TCP).

14. The confectionery product according to claim 10, wherein the capsules comprise Tricalcium Phosphate (TCP) and Zinc.

15. The confectionery product according to claim 10, wherein the particles are adapted for a slow release at least in one of the oral cavity or gastrointestinal tract of the ingredients comprised in the particles.

16. The confectionery product according to claim 1, wherein the particles size is distributed between about 1 μm and about 1000 p.m.

17. The confectionery product according to claim 1, wherein the particles are similar in size and/or shape.

18. The confectionery product according to claim 1, wherein the particles are different in size and/or shape.

19. The confectionery product according to claim 1, wherein the product does not induce a gag reflex when inserted into the oral cavity.

20. The confectionery product according to claim 1 further adapted to treat an additional oral cavity condition, selected from a group consisting of: candidiasis, bacterial infection, viral infection, burning mouth syndrome (BMS), dry mouth syndrome (xerostomia), abscess, mouth ulcers, gum inflammation, tooth decay and aphtha.
